(19) United States
(12) Patent Application Publication
(10) Pub. No.: US 2025/0357005 A1
(43) Pub. Date: Nov. 20, 2025

(54) LLMS FOR TIME SERIES PREDICTION IN MEDICAL DECISION MAKING

(71) Applicant: NEC Laboratories America, Inc., Princeton, NJ (US)

(72) Inventors: Wenchao Yu, Plainsboro, NJ (US); Wei Cheng, Princeton Junction, NJ (US); Haifeng Chen, West Windsor, NJ (US); Geon Lee, Monmouth Junction, NJ (US)

(21) Appl. No.: 19/205,111

(22) Filed: May 12, 2025

Related U.S. Application Data
(60) Provisional application No. 63/647,347, filed on May 14, 2024.
(60) Provisional application No. 63/649,615, filed on May 20, 2024.

(51) Int. Cl. G16H 50/20

(52) U.S. Cl. CPC G16H 50/20

(57) ABSTRACT

Methods and systems for time series analysis include generating a text summary of a time series using a first large language model (LLM) agent. A prompt is generated using a multi-modal encoder with the time series and the text summary as inputs. An event prediction is generated using a second LLM agent with the text summary and the prompt as inputs. An action is performed responsive to the event prediction.

### RELATED APPLICATION INFORMATION

This application claims priority to U.S. Patent Application No. 63/647,347, filed on May 14, 2024, and to U.S. Patent Application No. 63/649,615, filed on May 20, 2024, each incorporated herein by reference in its entirety.

### BACKGROUND

### Technical Field

The present invention relates to time series analysis and, more particularly, to the use of large language models (LLMs) in time series analysis.

### Description of the Related Art

Time series data provides a series of measurements that are taken at different points in time. Analysis of time series data has a wide variety of applications, including climate modeling, energy management, and healthcare monitoring. Machine learning models can be trained to analyze time series data, but specialized models may be limited in how they approach the contextual information that may be available for a given time series.

While LLMs can exhibit good performance on natural language processing tasks, they have limited applicability to time series data due to the fact that time series data is fundamentally distinct from the natural language training data used to create an LLM. Providing natural language contextual information in a zero-shot manner has proven to be limited by the overly simple contextualization of the time series data.

### SUMMARY

A method for time series analysis includes generating a text summary of a time series using a first large language model (LLM) agent. A prompt is generated using a multi-modal encoder with the time series and the text summary as inputs. An event prediction is generated using a second LLM agent with the text summary and the prompt as inputs. An action is performed responsive to the event prediction.

A system for time series analysis includes a hardware processor and a memory that stores a computer program. When executed by the hardware processor, the computer program causes the hardware processor to generate a text summary of a time series using a first large language model (LLM) agent, to generate a prompt using a multi-modal encoder with the time series and the text summary as inputs, to generate an event prediction using a second LLM agent with the text summary and the prompt as inputs, and to perform an action responsive to the event prediction.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram illustrating time-series analysis using a multi-modal encoder, in accordance with an embodiment of the present invention;

FIG. 2 is a block diagram illustrating the operation of a multi-modal encoder, in accordance with an embodiment of the present invention;

FIG. 3 is block/flow diagram illustrating the training and use of a multi-modal encoder in analyzing time series data, in accordance with an embodiment of the present invention;

FIG. 4 is a block diagram of a healthcare facility that performs multi-modal time series analysis, in accordance with an embodiment of the present invention;

FIG. 5 is a block diagram of a computing device that can train and use a model for time series analysis and event prediction, in accordance with an embodiment of the present invention;

FIG. 6 is a diagram of an exemplary neural network architecture that can be used to implement part of a multi-modal encoder, in accordance with an embodiment of the present invention; and

FIG. 7 is a diagram of an exemplary deep neural network architecture that can be used to implement part of a multi-modal encoder, in accordance with an embodiment of the present invention.

### DETAILED DESCRIPTION OF PREFERRED EMBODIMENTS

Time series data may be processed and analyzed by a large language model (LLM) by using the LLM not only as a predictor, but also as a contextualizer for the time series data. Two independent LLM agents may be used, where a first agent generates a textual summary with a comprehensive contextual understanding of the input time series data, and where a second agent uses the summary to make informed predictions of future events (e.g.,. anomalies). By contextualizing the time series data in this way, predictive performance is improved compared to directly prompting an LLM with raw time series data or its parameterized embedding.

Text summaries may further be leveraged as an augmentation to the time series data. A multi-modal encoder is trained to predict events and to learn representations using both the textual summaries and the raw time series data. The representations learned by the multi-modal encoder can be used to select relevant text summaries from a training set, which are provided as in-context examples to augment the prompt for the second LLM agent. This mutual enhancement, where the first LLM agent provides the encoder with contextualized information and where the enriched encoder supplies in-context examples to the second LLM agent, significantly improves overall performance. The present embodiments may further provide interpretable rationales for the predictions, addressing a need for transparency from machine learning models.

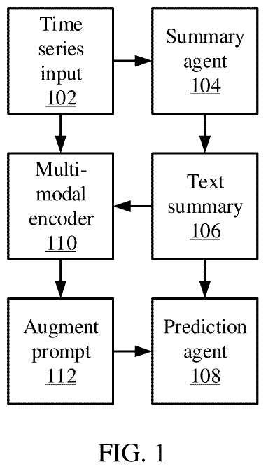

Referring now to FIG. 1, an overview of time series analysis is shown. The time series input 102 may include one or more series of measurements taken over a period of time. Each measurement may be continuous or integer-valued. In some examples, the measurements may relate to a health condition of a patient, for example from sensors that measure blood pressure, heart rate, blood oxygen saturation, etc. Integer-valued measurements may relate to categorical states, for example indicating whether a patient is conscious or unconscious.

The time series input 102 is processed by a first LLM, the summary agent 104. The summary agent 104 generates a natural language text summary 106 of the time series input 102 that contextualizes the information and that can be readily understood by an LLM. The summary agent 104 itself may be implemented using an LLM.

The text summary 106 used by multi-modal encoder 110, along with the raw time series input 102, to generate an augment prompt 112. The multi-modal encoder may use a multi-head self-attention to generate in-context examples and a prediction. The augment prompt 112 is combined with the text summary 106 as an input to a second LLM, the prediction agent 108, which performs a prediction task.

A pre-trained LLM [symbol] , parameterized by θ, may be pre-trained on an extensive corpus of natural language training data. The LLM may be employed in a zero-shot manner by keeping θ fixed, without any parameter updates or gradient computations. The LLM takes data D and optional supplementary data S to enhance the understanding of D and generate a more effective response R. Using a prompt generation function p, a prompt p(D,S) may be constructed. The inference of the LLM can be expressed as R= [symbol] (p(D,S)).

In this context, an LLM agent may be a specialized instance of [symbol] that is designed to perform a specific task. Each LLM agent, including the summary agent 104 and the prediction agent 108, is tailored to leverage its pre-trained domain knowledge to address different aspects of time series event prediction. Their roles are determined by distinct prompt functions, such as predicting or summarizing the given data.

Given a time series x=(x₁, . . . , x_{L}), where L is the number of past timesteps and each xₜ∈ [symbol] represents data from C channels at timestep t, the goal of time series event prediction is to predict an outcome y of a future event. Real-world time series data may be associated with contextual information derived from domain knowledge. For example, a patient's health measurements exist within the context of established medical knowledge. This contextual information helps to provide accurate future event predictions. The problem may be understood as a multi-class classification task.

In some embodiments, the contextual information associated with time series data may be used to enhance the comprehension and predictive capabilities of LLMs in a zero-shot manner. The summary agent 104 may be expressed as [symbol] _{C} and contextualizes the time series input 102, while the prediction agent 108 may be expressed as [symbol] _{P} and performs event prediction. The summary agent 104 generates a textual summary sₓ that contains the underlying context of the given time series x by leveraging its domain knowledge:

$${s}_{x} = {𝒜}_{c} ( x ) = {ℳ}_{θ} ( {p}_{C} ( x ) )$$

where (x) is a prompt that instructs the LLM to contextualize x. The generated summary sₓ includes relevant contextual insights beyond the raw time series data x.

The prediction agent 108 then uses sₓ to make informed event predictions:

$${\hat{y}}_{LLM} = {𝒜}_{P} ( {s}_{x} ) = {ℳ}_{θ} ( {p}_{P} ( {s}_{x} ) )$$

where p_{P}(sₓ) is a prompt that instructs the LLM to predict the outcome of an event based on sₓ. By incorporating the context-informed summary generated by [symbol] _{C}, [symbol] _{P} can account for the broader context. This dual-agent approach consistently outperforms a single-agent approach where the LLM directly predicts the event from the input time series data.

This framework is extended by the use of the multi-modal encoder 110, which synergizes with the LLM agents by introducing dual augmentations (e.g., the time series input 102 and the augment prompt 112). The multi-modal encoder 110 and the LLM agents complement each other to improve prediction accuracy.

The trainable multi-modal encoder 110 may be expressed as ε_{ϕ}, parameterized by ϕ. This encoder aims to capture intricate dynamic patterns in time series data more effectively than would a zero-shot LLM. In addition to times series x, the encoder 110 incorporates the textual summary sₓ generated by [symbol] _{C}. The encoder ϕ_{ϕ} generates its own prediction ŷ_{MM} and an embedding z of the multi-modal input (x, sₓ) as:

$$( {\hat{y}}_{MM} , z ) = {ℰ}_{ϕ} ( x , {s}_{x} )$$

where z is used to sample in-context examples. The multi-modal encoder 110 includes a language model that embeds text into a latent space and further includes a transformer encoder that captures dependencies between two modalities, as shown in greater detail below.

Once the multi-modal encoder is trained, it aids [symbol] _{P} in making more informed predictions by sampling relevant text summaries from the training set as demonstrations. Given the embedding z of the multi-modal input (x, sₓ), k summaries may be retrieved from the training set whose embeddings are closest to z. The training set may be expressed as [symbol] , while [symbol] ∈ [symbol] represents a set of embeddings of the training samples generated by ε_{ϕ}. The k pairs of text summaries and their corresponding outcomes are retrieved as the nearest neighbors of z in the embedding space as follows:

$$S = \{ ( {s}_{{x}_{j}} , {y}_{j} ) : j ∈ {NN}_{k} ( z ) \}$$

where NNₖ(z)=argtop [symbol] (−∥z− [symbol] ⱼ∥). These summaries and their outcomes are used as in-context examples for [symbol] _{P} to predict the outcome of sₓ as follows:

$${\hat{y}}_{LLM} = {𝒜}_{P} ( {s}_{x} , S ) = {ℳ}_{θ} ( {p}_{P} ( {s}_{x} , S ) )$$

The examples help the agent [symbol] _{P} better understand the time series input 102 by comparing the summaries and reasoning based on them.

The predictions from the multi-modal encoder 110 (ŷ_{MM}) and the prediction agent 108 (ŷ_{LLM}) are integrated through a linear combination: ŷ=λŷ_{LMM}+(1−λ)(ŷ_{MM}, where λ=[0,1] is a hyperparameter. Given that ŷ_{LLM} is discrete, it may be converted into a one-hot vector so that it can be fused with the continuous logit ŷ_{MM}. This fusion leverages complementary information from both models, enhancing the overall performance.

The prediction ŷ is interpretable by the introduction of two variants of the prompt function p_{P} used in [symbol] p. The variants,

$${𝒜}_{P}^{I} and {𝒜}_{P}^{E}$$

provide implicit interpretations and explicit interpretations, respectively, to enable distinct interpretations that enhance transparency.

For the implicit interpretation, the LLM [symbol] is prompted to generate a prediction and its corresponding rationale:

$$( {\hat{y}}_{LLM} , r ) = {𝒜}_{P}^{I} ( {s}_{x} , S ) = {ℳ}_{θ} ( {p}_{P}^{I} ( {s}_{x} , S )$$

where

$${p}_{P}^{I}$$

is a prompt function that instructs [symbol] to predict the event (ŷ_{LLM}) and also to provide the rationale r behind the prediction. The rationale leverages the LLM's domain knowledge and reasoning capabilities. In-context examples S are optional, but there inclusion leads to distinct implicit interpretations.

For the explicit interpretation, the LLM [symbol] is prompted to identify the most useful or relevant example from the in-context set S:

$$( {\hat{y}}_{LLM} , {s}_{{x}_{j}} ) = {𝒜}_{P}^{E} ( {s}_{X} , S ) = {ℳ}_{θ} ( {p}_{P}^{E} ( {s}_{x} , S )$$

where

$${p}_{P}^{E}$$

is a prompt function that instructs [symbol] to predict the event (ŷ_{LLM}) and to select the most relevant example (sₓ_{ⱼ}) from S. The input time series x can be compared with the corresponding time series xⱼ for further analysis.

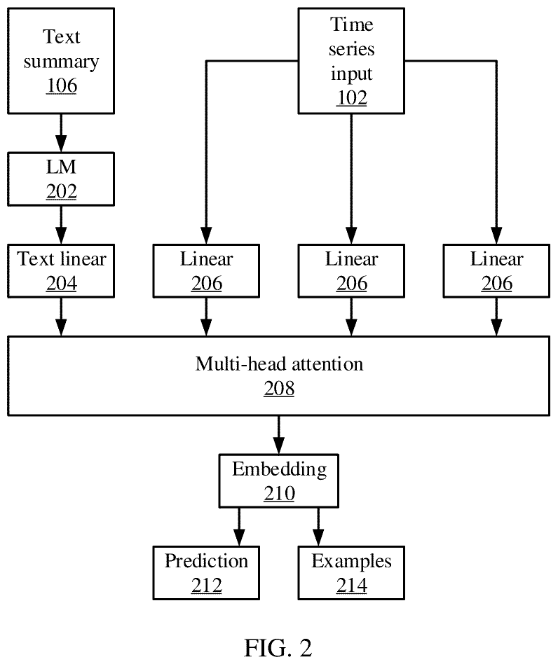

Referring now to FIG. 2, additional detail on the multi-modal encoder 110 is shown. The text summary 106 (sₓ) is processed by a pre-trained language model LM 202, which may have fewer parameters than the LLM [symbol] and which is therefore easier to fine-tune. The LM 202 represents the text summary sₓ as \hat{z}=LM(sₓ)∈ [symbol] , leveraging the output classification token embedding, where d′ denotes the fixed output dimension of the language model 202. This representation is then projected as \tilde{z}ₜₑₓₜ=\hat{z}Wₜₑₓₜ∈ [symbol] using a linear layer 204 represented as Wₜₑₓₜ∈ [symbol] . All time-series patches are independently embedded into vectors of dimension d, the common model feature size. Projecting \hat{z} to \tilde{z}ₜₑₓₜ places it in the same d-dimensional space as the time-series patch embeddings, allowing \tilde{z}ₜₑₓₜ to be concatenated with those patch vectors and fed directly into the multi-head attention block 208.

For the time series input 102, the time series x⁽ⁱ⁾∈x of a i^{th} channel is segmented into N overlapping patches \hat{x}⁽ⁱ⁾∈ [symbol] with patch length Lₚ and stride Lₛ, where

$$N = ⌈ \frac{L - {L}_{p}}{{L}_{s}} ⌉ + 1$$

holds. These patches are projected as

$${\tilde{z}}_{time}^{( i )} = {\hat{x}}^{( i )} {W}_{time}$$

∈ [symbol] using linear layers 206 represented as Wₜᵢₘₑ∈ [symbol] .

For each i^{th} channel of the time series input 102, the time series patch embeddings

$${\tilde{z}}_{time}^{( i )}$$

are concatenated with the text embedding \tilde{z}ₜₑₓₜ to construct

$${\tilde{z}}^{( i )} = [ {\tilde{z}}_{time}^{( i )} ; {\tilde{z}}_{text} ]$$

∈ [symbol] . Multi-head attention 208 captures relationships within this combined representation. For each attention head h∈{1, . . . , H}, a query is computed as:

$${Q}_{h}^{( i )} = {\tilde{z}}^{( i )} {W}_{h}^{Q}$$

a key is computed as:

$${K}_{h}^{( i )} = {\tilde{z}}^{( i )} {W}_{h}^{K}$$

and a value is computed as:

$${V}_{h}^{( i )} = {\tilde{z}}^{( i )} {W}_{h}^{V}$$

where

$${W}_{h}^{Q} , {W}_{h}^{K} , {W}_{h}^{V}$$

∈ [symbol] . Each hth attention head may be defined as:

$${z}_{h}^{( i )} = Softmax ( \frac{{Q}_{h}^{( i )} {K}_{h}^{{( i )}^{T}}}{\sqrt{d / H}} ) {V}_{h}^{( i )}$$

The outputs of the attention heads are aggregated and projected as

$${z}^{( i )} = [ {z}_{1}^{( i )} ; … ; {z}_{H}^{( i )} ] {W}^{H} ∈ {ℝ}^{d}$$

where W^{H}∈ [symbol] . The channels are flattened into a single embedding vector 210:

$$z = [ {z}^{( 1 )} , … , {z}^{( C )} ] ∈ {ℝ}^{d C}$$

A final linear layer W^{P}∈ [symbol] is applied to z to obtain a K-dimensional prediction logit 212, expressed as ŷ_{MM}=zW^{P}∈ [symbol] . The parameters ϕ are fine-tuned, including those of the LM 202 and the transformer encoder using cross-entropy loss. The in-context examples 214 are sampled as described above.

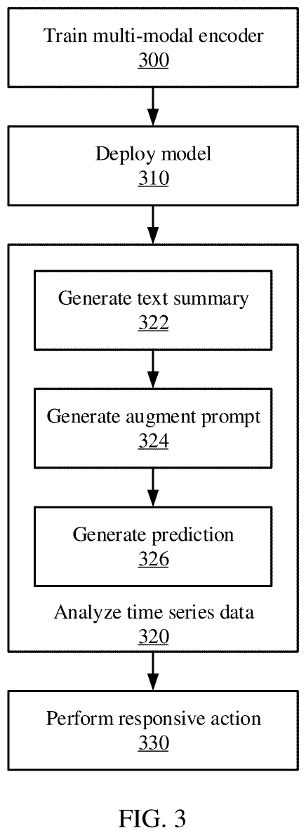

Referring now to FIG. 3, a method of training and using a multi-modal encoder 110 for use in time series analysis is shown. Block 300 trains the multi-modal encoder 110. Training may use a corpus of labels (x, s_{X}, y), where x is a multivariate time series, sₓ is a contextualized text summary produced by the summary agent [symbol] _{C}, and y is a ground-truth clinical outcome. The time series x may include data such as lab panels, blood pressure measurements, heart rate measurements, and ventilator settings. The ground-truth outcomes y may include an indication of fatality, sepsis onset, and positive or negative tests. During training of the encoder ε_{ϕ} with the input (x, s_{X}), it returns:

$$( {\hat{y}}_{MM} , z ) = {ℰ}_{ϕ} ( x , {s}_{x} )$$

where ŷ_{MM} is the prediction and z is the latent embedding used later for in-context retrieval. The training 300 may be performed using a cross-entropy loss as an optimization objective.

One the multi-modal encoder 110 has been trained, it may be deployed to a system where time series analysis is performed. Deployment may include copying the parameters of the trained multi-modal encoder 110 to the system where it can be executed on new time series data. The pre-trained LLM that implements the summary agent 104 and the prediction agent 108 may additionally be installed on, or called from, the system. In embodiments where the time series analysis will be performed at the same system as the training, the deployment 310 may be omitted.

Block 320 analyzes new time series data, for example collected from sensors that measure a patient's health state. Block 322 uses the summary agent 104 to generate a text summary 106 of the new time series data. Block 324 uses the trained multi-modal encoder 110 to generate augment prompt 112 using the text summary 106 and the new time series data. Block 326 then generates an event prediction using the prediction agent 108, based on the text summary 106 and the augment prompt 112.

Based on the event prediction generated by the time series data analysis 320, block 330 performs an action responsive to the predicted event. For example, the event prediction may relate to a health care event for a patient, and so the responsive action may include a treatment that prevents or mitigates the harm of the healthcare event. In some cases, the responsive action may include the automatic administration of an appropriate pharmaceutical, for example through an intravenous supply.

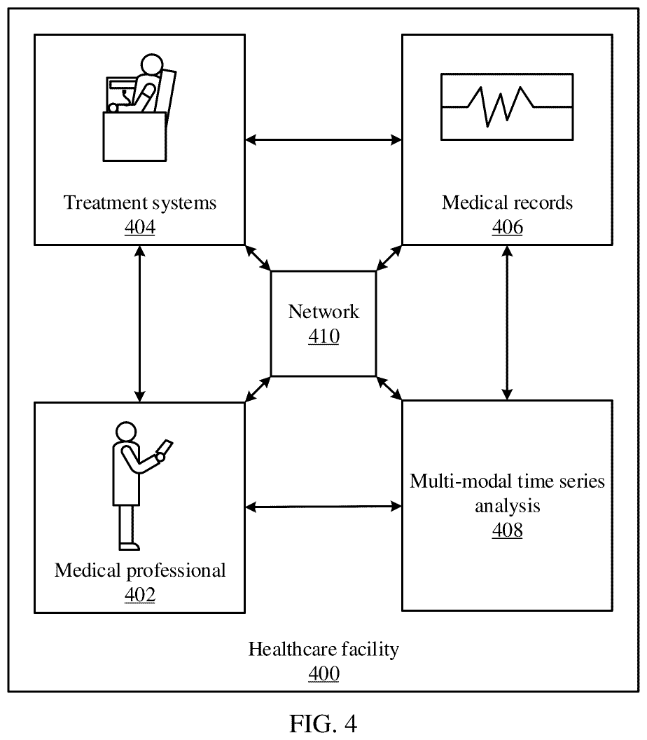

Referring now to FIG. 4, a diagram of time series analysis is shown in the context of a healthcare facility 400. Multi-modal time series analysis 408 may be used to analyze the time series measurements from a patient to help predict future healthcare events and identify treatments. Multi-modal time series analysis 408 may be used to generate a treatment responsive to a patient's medical condition based on up-to-date medical records 406.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

Medical professionals 402 may use multi-modal time series analysis 408 to provide rapid-response healthcare that is tailored to the patient's needs. For example, the medical professionals 402 may use multi-modal time series analysis 408 to identify emerging health conditions based on real-time time series data from a patient, which can be used to aid in medical decision making and to identify and administer appropriate treatments.

The different elements of the healthcare facility 400 may communicate with one another via a network 410, for example using any appropriate wired or wireless communications protocol and medium. Thus the multi-modal time series analysis 408 can be used to identify a patient's specific condition and select a treatment, for example using measurements and medical records 406. The treatment systems 404 may be used to automatically administer a treatment, for example by administration of a pharmaceutical via an intravenous supply, responsive to the prediction of a particular healthcare event.

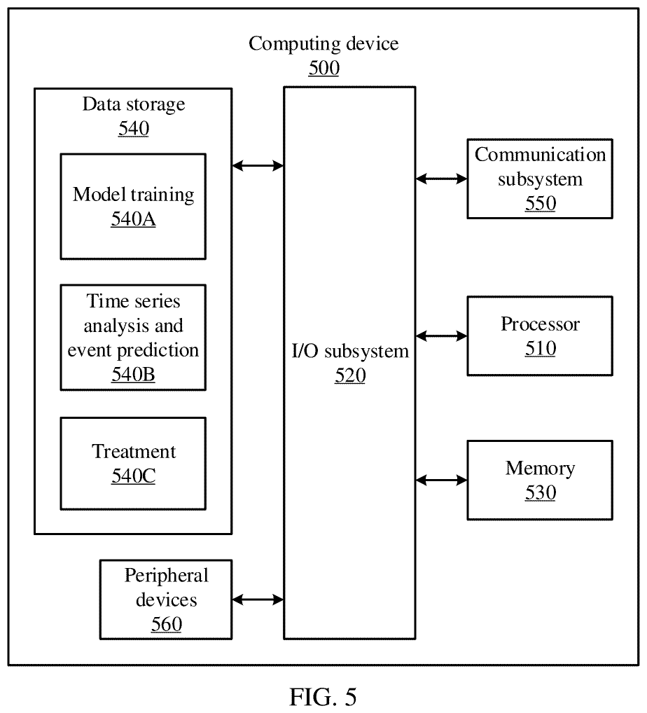

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for training a multi-modal encoder, 540B for performing time series analysis and event prediction, and/or 540C for administering a treatment. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

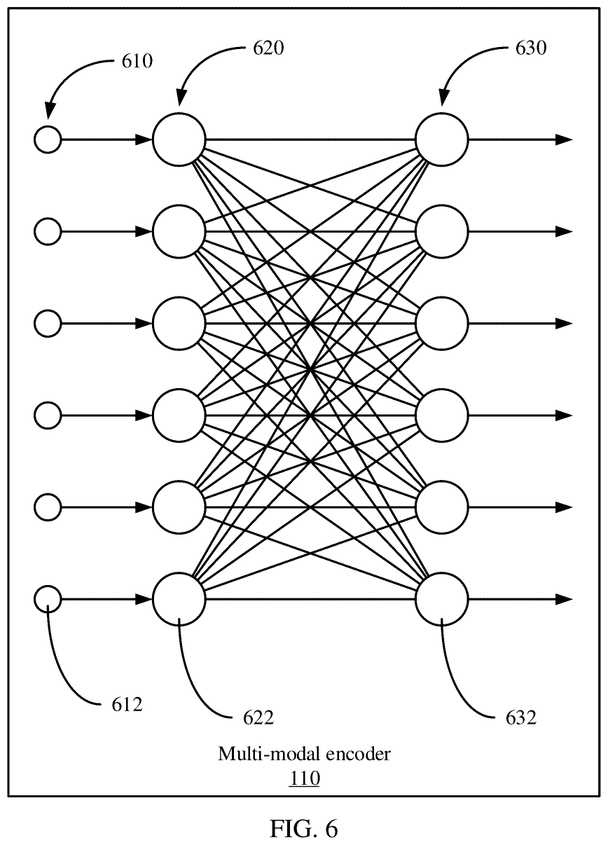

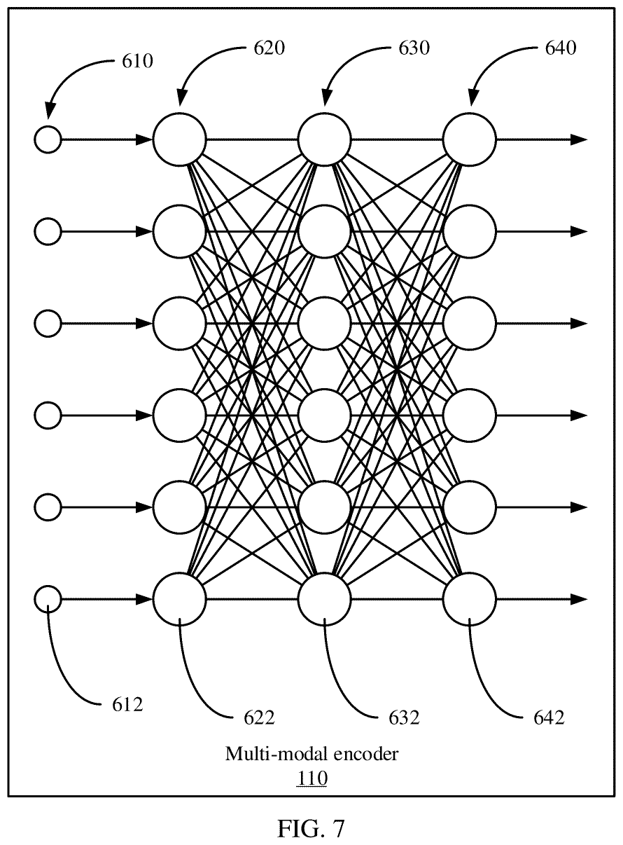

Referring now to FIGS. 6 and 7, exemplary neural network architectures are shown, which may be used to implement parts of the present models, such as the multi-modal encoder 110. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor-or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for time series analysis, comprising:
generating a text summary of a time series using a first large language model (LLM) agent;
generating a prompt using a multi-modal encoder with the time series and the text summary as inputs;
generating an event prediction using a second LLM agent with the text summary and the prompt as inputs; and
performing an action responsive to the event prediction.

2. The method of claim 1, wherein the first LLM agent and the second LLM agent are implemented using respective prompts to a same LLM.

3. The method of claim 2, wherein the multi-modal encoder is implemented using a language model having fewer parameters than the LLM.

4. The method of claim 1, wherein the multi-modal encoder concatenates an embedding of a classification of the text summary with embeddings of patches of the time series to generate a concatenated embedding.

5. The method of claim 4, wherein the multi-modal encoder processes the concatenated embedding with a multi-head attention and flattening an output of the multi-head attention to create an embedded output.

6. The method of claim 5, wherein the multi-modal encoder uses a linear layer to convert the embedded output into a K-dimensional prediction logit as part of the prompt.

7. The method of claim 5, wherein the multi-modal encoder samples a training dataset to select an in-context example for the prompt using the embedded output.

8. The method of claim 1, wherein the multi-modal encoder is implemented as a machine learning model.

9. The method of claim 1, wherein the time series includes measurements of a patient's health condition for medical decision making.

10. The method of claim 9, wherein the action includes automatic administration of treatment based on the event prediction relating to a health event.

11. A system for time series analysis, comprising:
a hardware processor; and
a memory that stores a computer program which, when executed by the hardware processor, causes the hardware processor to:
generate a text summary of a time series using a first large language model (LLM) agent;
generate a prompt using a multi-modal encoder with the time series and the text summary as inputs;
generate an event prediction using a second LLM agent with the text summary and the prompt as inputs; and
perform an action responsive to the event prediction.

12. The system of claim 11, wherein the first LLM agent and the second LLM agent are implemented using respective prompts to a same LLM.

13. The system of claim 12, wherein the multi-modal encoder is implemented using a language model having fewer parameters than the LLM.

14. The system of claim 11, wherein the multi-modal encoder concatenates an embedding of a classification of the text summary with embeddings of patches of the time series to generate a concatenated embedding.

15. The system of claim 14, wherein the multi-modal encoder processes the concatenated embedding with a multi-head attention and flattening an output of the multi-head attention to create an embedded output.

16. The system of claim 15, wherein the multi-modal encoder uses a linear layer to convert the embedded output into a K-dimensional prediction logit as part of the prompt.

17. The system of claim 15, wherein the multi-modal encoder samples a training dataset to select an in-context example for the prompt using the embedded output.

18. The system of claim 11, wherein the multi-modal encoder is implemented as a machine learning model.

19. The system of claim 11, wherein the time series includes measurements of a patient's health condition for medical decision making.

20. The system of claim 19, wherein the action includes automatic administration of treatment based on the event prediction relating to a health event.
